(19) United States
(12) Patent Application Publication
(10) Pub. No.: US 2004/0133019 A1
(43) Pub. Date: Jul. 8, 2004

(54) Catalyst for the oxidation of hydrocarbons to epoxides

(76) Inventors: Markus Dugal, Kempen (DE); Markus Weisbeck, Koln (DE); Gerhard Wegener, Mettmann (DE)

Correspondence Address: BAYER POLYMERS LLC, 100 BAYER ROAD, PITTSBURGH, PA, 15205, US

(21) Appl. No.: 10/683,741

(22) Filed: Oct. 10, 2003

(30) Foreign Application Priority Data
Oct. 14, 2002 (DE) 10247784.1

(51) Int. Cl. B01J 23/88; C07D 301/06

(52) U.S. Cl. 549/533; 502/317

(57) ABSTRACT

The present invention relates to a catalyst containing gold in elemental or bound form and molybdenum in oxidation state +VI, and to a process for the oxidation of a hydrocarbon containing at least one double bond to an epoxide, which process involves reacting the hydrocarbon with oxygen in the presence of hydrogen and in the presence of the catalyst according to the invention.

### FIELD OF THE INVENTION

The present invention relates to a catalyst containing gold in elemental or bound form and molybdenum in oxidation state +VI, and to a process for the oxidation of a hydrocarbon containing at least one double bond to an epoxide, which process involves reacting the hydrocarbon with oxygen in the presence of hydrogen and in the presence of the inventive catalyst.

### BACKGROUND OF THE INVENTION

Epoxides are important raw materials for the polyurethane industry. There are a number of processes for their preparation, some of which have also been converted to an industrial scale.

EP-A 0 933 130 discloses the preparation of ethylene oxide by reaction of ethene with air or with gas mixtures containing oxygen, in the presence of a catalyst containing silver. That process is also called direct oxidation.

In order to prepare epoxides having more than two carbon atoms, it is usual on an industrial scale to use hydrogen peroxide, organic hydroperoxides or hypochlorite as oxidizing agent in the liquid phase. EP-A 0 930 308 discloses the use of ion-exchanged titanium silicalites as catalyst for that reaction.

Another class of catalysts which allow propene to be oxidized in the gas phase to propene oxide is disclosed in EP-A 0 709 360 (equivalent to U.S. Pat. No. 5,623,090). According to the teaching of EP-A 0 709 360, gold on titanium dioxide is used as catalyst. Oxygen in the presence of hydrogen is used as oxidizing agent. The catalysts according to EP-A 0 709 360 are distinguished by high selectivity (over 95%). However, the yields are low and the lifetime of the catalysts is short even under mild reaction conditions (normal pressure, low temperature).

DE-A 199 59 525 describes a marked improvement in the yields and useful lives with the use of gold-containing and titanium-containing Si—H-modified inorganic-organic hybrid materials, although significant deactivation (over 7% in 10 days under normal pressure) is still to be found.

As an alternative to titanium, a large number of other elements can also be used in conjunction with gold in order to obtain active catalysts for the epoxidation of propene in the gas phase with oxygen and hydrogen. EP-A 1 125 632 and EP-A 1 125 933 (equivalent to WO 01/58887) disclose combinations of gold with Sc, Y, La, Zr, Hf, V, Nb, Ta, Cr, Mo, W or lanthanoid-containing carrier materials. However, those combinations generally provide only very poor yields (less than 0.1% conversion to propene oxide (PO)). In addition, some of those catalysts exhibit poor selectivities. In the case of Au/Mo systems, only selectivities of less than 75% at conversions of 0.01%, based on 6% propene in the feed gas, are described. Feed gas is understood as being the gas mixture that is supplied to the reaction. The feed gas contains propene, oxygen and hydrogen. No information is given in EP-A 1 125 632 and EP-A 1 125 933 regarding the lifetime of the catalyst.

The titanium-free gold-metal catalysts known from the prior art have considerable disadvantages compared with the titanium-containing catalysts known from the prior art.

### SUMMARY OF THE INVENTION

The present invention accordingly provides catalysts for the oxidation of a hydrocarbon containing at least one double bond, with oxygen, in the presence of hydrogen, to an epoxide.

### BRIEF DESCRIPTION OF THE FIGURES

The present invention will now be described for purposes of illustration and not limitation in conjunction with the figures, wherein:

FIG. 1 shows the TEM image of a catalyst obtained according to Example 7, on a scale of 100,000:1; and

FIG. 2 illustrates the XAS spectra of four catalysts obtained according to the Examples.

### DETAILED DESCRIPTION OF THE INVENTION

The present invention will now be described for purposes of illustration and not limitation. Except in the operating examples, or where otherwise indicated, all numbers expressing quantities, percentages, and so forth in the specification are to be understood as being modified in all instances by the term “about.”

The catalyst of the present invention contains gold in elemental or bound form and molybdenum in oxidation state +VI.

The present invention also provides a process for the preparation of the inventive catalyst involving,

a) application of gold in elemental or bound form to a carrier matrix,

b) application of molybdenum to the carrier matrix, and

c) tempering of the carrier matrix loaded with gold and molybdenum at a temperature of from 200 to 500° C., largely with the exclusion of oxygen.

The present invention also provides the catalyst obtainable in accordance with the disclosed process.

The present invention also provides a process for the oxidation of a hydrocarbon containing at least one double bond to an epoxide, which process involves reacting the hydrocarbon with an oxygen source in the presence of a hydrogen source and in the presence of the catalyst according to the invention.

In an embodiment of the present invention, the oxygen source in that process is molecular oxygen (O₂).

In an embodiment of the present invention, the hydrogen source in that process is molecular hydrogen (H₂).

The catalyst according to the invention contains molybdenum of oxidation state +VI. It is, of course, also possible for molybdenum in other oxidation states additionally to be present. However, at least part of the total molybdenum present is in oxidation state +VI. The catalytic activity is thereby ensured.

In an embodiment of the present invention, the molybdenum is present in the catalyst in octahedral coordination. That means that catalytically active centers of molybdenum in oxidation state +VI are present. It is, of course, also possible for further molybdenum additionally to be present in non-octahedral coordination. The coordination of the molybdenum can be determined by XAS measurements, for example. XAS stands for X-ray absorption spectroscopy. The XAS measurements are explained in detail in the Examples. Octahedral coordination is understood as meaning both regular and distorted octahedral coordination.

In one embodiment of the present invention, the catalyst according to the invention is substantially free of titanium. The expression “substantially free of titanium” means that titanium is present in the catalyst at most in a small amount as an impurity, but does not make any substantial contribution towards the catalytic activity.

A major disadvantage of catalysts containing titanium is their tendency to deactivate, that is to say decreasing activity as the operating time increases.

The catalysts according to the invention are suitable for the oxidation of any desired hydrocarbons containing at least one double bond. Olefins are preferred, and propene is particularly preferred.

The catalysts according to the invention have many advantages. The yield, the productivity and the selectivity in the oxidation of hydrocarbons containing at least one double bond, with gas mixtures containing oxygen and hydrogen, to epoxides in the presence of the catalysts according to the invention are high.

The lifetime of the catalysts according to the invention at high reaction temperatures, for example over 150° C., is long.

The catalysts according to the invention, while having high selectivity and activity for propene oxide formation, have a long catalyst lifetime at reaction conditions under normal pressure and, especially, under elevated pressure.

In an embodiment of the present invention, the catalyst according to the invention is prepared by a process that includes at least one tempering step at from 200 to 500° C., largely with the exclusion of oxygen. Largely with the exclusion of oxygen means an oxygen content of preferably less than 5 vol. %, more preferably less than 2 vol. %, and most preferably less than 1 vol. %, in the atmosphere above the catalyst during the tempering.

In an embodiment of the present invention, the catalyst according to the invention contains molybdenum that is bonded chemically or physically to the surface of or inside a carrier matrix. The carrier matrix may consist of oxides of main group elements or oxides of subsidiary group elements, such as, for example, silicon, aluminum, zirconium, zinc, magnesium, calcium, tantalum, niobium, molybdenum or manganese oxides, which may additionally contain organic groupings, such as, for example, alkyl, alkoxy, cycloalkyl, fluoroalkyl, aryl, allyl or vinyl groups, or of corresponding hydroxides or carbonates, but it is not limited thereto.

Particular preference is given to the use of organically modified or unmodified silicon oxides as carrier matrices. As starting materials for their preparation there may be used, inter alia, silicon alkoxides, such as, for example, tetramethoxysilane or tetraethoxysilane, which are reacted by hydrolysis and condensation under from acids, such as, for example, HNO₃, HI, HCl, HBr, HF, H₂SO₄, formic acid, acetic acid, trifluoroacetic acid, toluenesulfonic acid, or bases, such as, for example, NaOH, KOH, Ca(OH)₂, NH₄OH or fluoride salts, such as NaF, NH₄F.

The mentioned organic groupings can, for example, be incorporated directly into the carrier matrix in a sol-gel process in the form of organically modified silicon alkoxides, such as, for example, methyltrimethoxysilane, methyltriethoxysilane, ethyltrimethoxysilane, propyltrimethoxysilane, butyltrimethoxysilane, octyltrimethoxysilane, dimethyldimethoxysilane, phenyltrimethoxysilane, trivinylmethoxysilane, trifluoropropyltrimethoxysilane, or they can be introduced by subsequent modification with organylating agents, such as, for example, trichloroalkylsilanes, dichlorodialkylsilanes, trialkylchlorosilanes or hexamethyldisilazane.

The specific surface area of the matrix, as is accessible to the person skilled in the art by N₂ physisorption measurements, should be large and should advantageously be >1 m²/g and preferably in the range from 10 to 1000 m²/g.

The carrier matrix may already contain gold at the time of binding of the molybdenum, but that is not a necessary condition.

The binding of the molybdenum to the carrier matrix can be carried out in many ways, such as, for example, by impregnation, co-precipitation, deposition-precipitation, sol-gel processes or vapor deposition, with or without subsequent thermal treatment, the mentioned examples being non-limiting.

Dependent, inter alia, on the method by which it is applied to the carrier, the molybdenum can be used in the solid, gaseous or dissolved form of a large number of starting compounds having different oxidation states from 0 to +VI. Non-limiting examples are MoCl₃, MoBr₃, MoCl₅, MoF₅, Mo(OC₃H₇)₅, Mo(OC₃H₅)₃, Mo(OC₂H₅)₅, K₂MoO₄, (NH₄)Mo₇O₁₈, MoO₃, bis-acetylacetonato-dioxo-molybdenum (VI) or mixtures of the mentioned compounds.

In an embodiment of the invention, the molybdenum is used in the form of an alkoxide in the acid- or base-catalyzed hydrolysis/condensation process of a sol-gel reaction of, for example, silicon alkoxides (reaction to SiO₂) and polymerized into or physically included in the sol-gel matrix in the form of oxidic molybdenum. In a further particular embodiment, the molybdenum is used in the form of an aqueous solution of MoO₃ (dissolved at pH 8) and applied to a pre-formed, porous carrier by impregnation.

Part of the molybdenum in the catalyst according to the invention must be in oxidation state +VI and have the coordination number 6 in octahedral coordination. The oxidation state and the coordination number of the molybdenum are derived from analytical information, as is accessible to the person skilled in the art by chemical-physical solids characterization methods, such as XPS (X-ray photoelectron spectroscopy), XAS (X-ray absorption spectroscopy) or TPO/TPR (temperature programmed oxidation/reduction).

If a molybdenum compound having an oxidation state of less than +VI is used, it is necessary to increase the oxidation state before the catalyst is used. That can be effected, for example, by calcination at a temperature preferably above 150° C. The total concentration of molybdenum in the catalyst can be as large or as small as desired, within wide limits. For optimum catalyst functioning, concentrations of from 0.1 to 10 mol. %, based on the carrier, are preferred, with concentrations of from 0.5 to 5 mol. % being particularly preferred.

In the case of silicate carriers, mol. % are defined as the molar proportion of molybdenum precursor compound in the number of moles of the carrier precursor (silicon alkoxide)+number of moles of molybdenum precursor compound (in %). This corresponds to the molar proportion of molybdenum in the totality of all central atoms in the solid carrier (silicon+molybdenum). The advantage of indicating the concentration in that manner is that it is possible, independently of the number of oxygen atoms (which in turn is dependent on the coordination number, which is poorly defined in the amorphous solid), to indicate the molybdenum content without having to carry out an elemental analysis.

In addition to molybdenum, the catalyst according to the invention also contains gold, which may likewise be bonded physically or chemically to the same carrier matrix at least partially in the form of nanostructured gold particles. The term “nanostructured” will be understood by the person skilled in the art to mean that the measure falls below 100 nm in at least one dimension.

The structure of the gold particles can be examined by methods of electron microscopy, such as, for example, transmission electron microscopy. The carrier matrix for the binding of the gold can be characterized by the description given above. The carrier matrix may already contain molybdenum before the gold is applied; however, that is not a necessary condition.

Binding of the nanostructured gold to the secondary matrix can be carried out by various methods, such as, for example, deposition-precipitation or co-precipitation processes, sol-gel inclusion processes, impregnation processes, CVD (chemical vapor deposition) or PVD (physical vapor deposition) or sputtering, but is preferably carried out by deposition-precipitation.

A very wide variety of gold compounds can be used as starting compounds for the binding, for example AuCl₃, HAuCl₄, gold(III) acetate, gold(III) nitrate, gold colloids, gold-amine complexes, gold-phosphane complexes or gold-thiol complexes.

In addition to the gold compound, organic auxiliary substances, such as, for example, oxalates or citrates, can be used. It is also possible to use inorganic acid, basic or neutral auxiliary substances, for example LiOH, NaOH, KOH, Ca(OH)₂, NH₄OH, H₂SO₄, H₃PO₄, Na₂(HPO₄), Na(H₂PO₄), NaHCO₃, Na₂CO₃ or HCl, for adjusting the pH or for buffering.

Where the preferred process of deposition-precipitation is used, preferably a pH range of from 4 to 9, more preferably a pH range of from 5 to 8.5, is adjusted.

The reaction temperature for the gold precipitation is advantageously in a range from 5 to 90° C., preferably in a range from 15 to 80° C.

The process for the preparation of the catalyst according to the invention preferably comprises tempering in a temperature range of from 200 to 500° C., preferably in the range from 250 to 400° C., largely with the exclusion of oxygen. To that end, the procedure can be carried out under an inert gas atmosphere, such as, for example, nitrogen or noble gases, or alternatively under other gases, such as, for example, H₂, CO, CO₂ or mixtures of those gases.

It is generally advantageous if any temperature treatment during the preparation of the catalyst, between the process of binding of the gold and the use as catalyst, takes place above 250° C., largely with the exclusion of oxygen, independently of whether the binding of the molybdenum has taken place before or after binding of the gold.

In the deposition-precipitation, only a portion of the gold available in the solution is generally deposited on the carrier. The concentration of gold on the carrier matrix, as can be determined by methods of elemental analysis, for example by atom absorption spectroscopy, is preferably below 5 wt. %, more preferably below 1 wt. %, based on the total weight of the catalyst.

In addition to the elements gold and molybdenum, any desired further elements, apart from titanium, from the main groups and subsidiary groups of the periodic system of the elements can be introduced into the catalyst as promoters by means of the above-mentioned processes for the binding of elements. Examples are Nb, Ta, Sc, Y, La, Cr, W, Hf, Zr, Re, Pd, Pt, Rh, Ir, Ag, Ru, Cu, lanthanides, alkali metals or alkaline earth metals, as well as halogens.

The catalyst according to the invention can be used in the epoxidation of hydrocarbons containing at least one double bond (for example ethene, propene, 1-butene, 2-butene, cis-2-butene, trans-2-butene, 1,3-butadiene, pentene, 1-hexene, hexadiene and cyclohexene).

The catalyst according to the invention can be used in any physical form, for example in the form of a ground powder, pellets, spherical particles, granules or extrudates.

The preferred use of the catalyst according to the invention is the catalytic gas-phase epoxidation of propene with oxygen in the presence of hydrogen.

The relative molar ratio of propene, oxygen, hydrogen and, optionally, a diluting gas in the mentioned process can be varied within wide limits. The molar amount of propene, relative to the total number of moles of propene, oxygen, hydrogen and diluting gas, can also be varied within wide limits. A molar excess of propene, based on the oxygen used, is preferably employed. The propene content is typically greater than 1 mol. % and less than 80 mol. %. Propene is preferably used in the range from 5 to 60 mol. %, more preferably from 10 to 50 mol. %.

The oxygen can be used in a very wide variety of forms. For example, molecular oxygen (O₂), air, ozone and/or a nitrogen oxide can be used as the oxygen source. Molecular oxygen is preferred.

The molar amount of oxygen, based on the total number of moles of propene, oxygen, hydrogen and diluting gas, can be varied within wide limits. The oxygen is preferably used in a smaller amount relative to the hydrocarbon. From 1 to 30 mol. %, more preferably from 5 to 25 mol. %, are used.

In the absence of hydrogen, the catalysts according to the invention exhibit only very slight activity and selectivity. Any known hydrogen source can be used for the mentioned epoxidation process. For example, molecular hydrogen (H₂), synthesis gas, or hydrogen from the dehydrogenation of hydrocarbons or alcohols can be used. The hydrogen may also be introduced into the reaction system in the form of a complex-bonded species, for example in the form of a catalyst-hydrogen complex. Molecular hydrogen is the preferred hydrogen source.

The molar amount of hydrogen, relative to the other reaction gases propene, oxygen and diluting gas, can be varied within wide limits. Typical hydrogen contents are greater than 0.1 mol. %, preferably from 4 to 90 mol. %, more preferably from 5 to 75 mol. %.

In addition to the essential starting gases described above, a diluting gas, such as nitrogen, helium, argon, methane, carbon dioxide or similar, predominantly inert gases, can optionally be used.

The gas-phase epoxidation using the catalysts according to the invention can be carried out over a wide temperature range. Preferably, temperatures of from 30° C. to 350° C., more preferably from 80° C. to 250° C. and most preferably from 120° C. to 210° C., are used.

The pressure, the amount of catalyst used and the gas flow rates can be varied as desired. In the case of pressure, the procedure is advantageously carried out in a range of from 0.1 bar to 100 bar, preferably in a range from 0.5 bar to 50 bar (absolute pressure). For process-related reasons, it is desirable to carry out the reaction under elevated pressure. It is therefore essential that the activity, selectivity and lifetime of the catalyst should be maintained under such conditions.

### EXAMPLES

The Examples which follow merely illustrate the invention. The invention is not limited in its scope to the Examples.

General procedure for testing the catalysts

Tubular metal reactors or tubular glass reactors having an inside diameter of 10 mm and a length of 20 cm were used, the temperature of the reactors being adjusted by means of an oil thermostat. The reactors were supplied with the starting gases (propene, oxygen and hydrogen) by way of three mass flow regulators (for propene, for oxygen and for hydrogen). 500 mg of catalyst were introduced for the reaction.

Unless mentioned otherwise, the tests were carried out under the chosen standard conditions: reaction temperature 170° C., catalyst load 2.8 liters per gram of catalyst and per hour (l/g(cat)*h)), gas composition (molar) propene/H₂/O₂=20/70/10. The reaction gases were subjected to quantitative analysis by gas chromatography (GC). Detection of the reaction products was carried out by means of a combined FID/TCD method (FID=flame ionization detector, TCD=thermal conductivity detector).

### Example 1

Example 1 describes the preparation and use of a catalyst of a molybdenum-containing silicon oxide which has been prepared by a sol-gel process and loaded with gold by means of a deposition-precipitation method.

3957 mg (26 mmol.) of tetramethoxysilane were placed in a polyethylene beaker (PE beaker) together with 3588 mg (78 mmol.) of ethanol. 710 mg (2.6 mmol.) of MoCl₅ were then added, with stirring. When the MoCl₅ had dissolved, 1638 mg (26 mmol.) of HNO₃ in a total of 468 mg (106 mmol.) of H₂O were added, and the batch was stirred until gelling occurred. After an ageing time of 12 hours, the gel was dried at 60° C. and 200 mbar, comminuted in a mortar and then calcined for 15 hours at 300° C. The molybdenum-containing carrier was introduced into a centrifuge vessel, and 7.5 ml of an aqueous solution of HAuCl₄ (concentration=1 g Au/l) were added. Sodium hydroxide solution (conc.=0.1 mol./l) was added, with stirring, until a constant pH value of 8.0±0.1 was maintained over a period of several minutes. 7.6 ml of an aqueous sodium citrate solution (concentration=0.015 mol./l, adjusted to pH 8) were then added, and stirring was carried out for 10 minutes. After centrifugation, washing was carried out 3 times using 20 ml of deionized water each time, followed by centrifugation. The powder loaded with gold was dried at 60° C./200 mbar and then tempered for 4 hours at 300° C. under an N₂ atmosphere. The catalyst was tested under standard conditions.

### Example 2

Example 2 describes the preparation and use of a catalyst analogously to Example 1, 10% of a methyl-substituted silicon alkoxide being used in the sol-gel process. 3561 mg (23.4 mmol.) of tetramethoxysilane and 354 mg of methyltrimethoxy-silane (2.6 mmol.) were placed in a PE beaker together with 3588 mg (78 mmol.) of ethanol. 710 mg (2.6 Mmol.) of MoCl₅ were then added, with stirring. When the MoCl₅ had dissolved, 1638 mg (26 mmol.) of HNO₃ in a total of 468 mg (106 mmol.) of H₂O were added, and the batch was stirred until gelling occurred. After an ageing time of 12 hours, the gel was dried at 60° C. and 200 mbar, comminuted in a mortar and then calcined for 15 hours at 300° C. The molybdenum-containing carrier was introduced into a centrifuge vessel, and 7.5 ml of an aqueous solution of HAuCl₄ (concentration 1 g Au/l) were added. Sodium hydroxide solution (conc.=0.1 mol./l) was added, with stirring, until a constant pH value of 8.0±0.1 was maintained over a period of several minutes. 7.6 ml of an aqueous sodium citrate solution (concentration=0.015 mol./l, adjusted to pH 8) were then added, and stirring was carried out for 10 minutes. After centrifugation, washing was carried out 3 times using 20 ml of deionized water each time, followed by centrifugation. The powder loaded with gold was dried at 60° C./200 mbar and then tempered for 4 hours at 300° C. under an N₂ atmosphere. The catalyst was tested under standard conditions.

### Example 3

Example 3 describes the preparation and use of a catalyst analogously to Example 2, a different molybdenum compound being used in the sol-gel process.

3561 mg (23.4 mmol.) of tetramethoxysilane and 354 mg of methyltrimethoxysilane (2.6 mmol.) were placed in a PE beaker together with 1656 mg (36 mmol.) of ethanol. 20.33 g of a 5% solution of Mo(V) isopropoxide in isopropanol (2.6 mmol. of Mo) were then added, with magnetic stirring. 1638 mg (26 mmol.) of HNO₃ in a total of 468 mg (106 mmol.) of H₂O were then added, and the batch was stirred until gelling occurred. After an ageing time of 12 hours, the gel was dried at 60° C./200 mbar, comminuted in a mortar and then calcined for 15 hours at 300° C. The molybdenum-containing carrier was introduced into a centrifuge vessel, and 7.5 ml of an aqueous solution of HAuCl₄ (concentration 1 g Au/l) were added. Sodium hydroxide solution (conc.=0.1 mol./l) was added, with stirring, until a constant pH value of 8.0±0.1 was maintained over a period of several minutes. 7.6 ml of an aqueous sodium citrate solution (concentration=0.015 mol./l, adjusted to pH 8) were then added, and stirring was carried out for 10 minutes. After centrifugation, washing was carried out 3 times using 20 ml of deionized water each time, followed by centrifugation. The powder loaded with gold was dried at 60° C. and 200 mbar and then tempered for 4 hours at 300° C. under an N₂ atmosphere. The catalyst was tested under standard conditions.

### Example 4

Example 4 describes the preparation and use of a catalyst analogously to Example 3, 10% of a propyl-substituted silicon alkoxide being used in the sol-gel process instead of the methyl-substituted silicon alkoxide.

3561 mg (23.4 mmol.) of tetramethoxysilane and 469 mg of propyltrimethoxysilane (2.6 mmol.) were placed in a PE beaker together with 1656 mg (36 mmol.) of ethanol. 20.33 g of a 5% solution of Mo(V) isopropoxide in isopropanol (2.6 mmol. of Mo) were then added, with stirring. 1638 mg (26 mmol.) of HNO₃ in a total of 468 mg (106 mmol.) of H₂O were then added, and the batch was stirred until gelling occurred. After an ageing time of 12 hours, the gel was dried at 60° C. and 200 mbar, comminuted in a mortar and then calcined for 15 hours at 300° C. The molybdenum-containing carrier was introduced into a centrifuge vessel, and 7.5 ml of an aqueous solution of HAuCl₄ (concentration 1 g Au/l) were added. Sodium hydroxide solution (conc.=0.1 mol./l) was added, with stirring, until a constant pH value of 8.0±0.1 was maintained over a period of several minutes. 7.6 ml of an aqueous sodium citrate solution (concentration=0.015 mol./l, adjusted to pH 8) were then added, and stirring was carried out for 10 minutes. After centrifugation, washing was carried out 3 times using 20 ml of deionized water each time, followed by centrifugation. The powder loaded with gold was dried at 60° C. and 200 mbar and then tempered for 4 hours at 300° C. under an N₂ atmosphere. The catalyst was tested under standard conditions.

### Example 5

Example 5 describes the preparation and use of a catalyst analogously to Example 1, 10% of a vinyl-substituted silicon alkoxide being used in the sol-gel process.

3561 mg (23.4 mmol.) of tetramethoxysilane and 365 mg of trivinylmethoxysilane (2.6 mmol.) were placed in a PE beaker together with 3588 mg (78 mmol.) of ethanol. 710 mg (2.6 mmol.) of MoCl₅ were then added, with stirring. When the MoCl₅ had dissolved, 1638 mg (26 mmol.) of HNO₃ in a total of 468 mg (106 mmol.) of H₂O were added, and the batch was stirred until gelling occurred. After an ageing time of 12 hours, the gel was dried at 60° C. and 200 mbar, ground in a mortar and then calcined for 15 hours at 300° C. The molybdenum-containing carrier was introduced into a centrifuge vessel, and 7.5 ml of an aqueous solution of HAuCl₄ (concentration 1 g Au/l) were added. Sodium hydroxide solution (conc.=0.1 mol./l) was added, with stirring, until a constant pH value of 8.0±0.1 was maintained over a period of several minutes. 7.6 ml of an aqueous sodium citrate solution (concentration=0.015 mol./l, adjusted to pH 8) were then added, and stirring was carried out for 10 minutes. After centrifugation, washing was carried out 3 times using 20 ml of deionized water each time, followed by centrifugation. The powder loaded with gold was dried at 60° C./200 mbar and then tempered for 4 hours at 300° C. under an N₂ atmosphere. The catalyst was tested under standard conditions.

### Example 6

This Example describes the use of the catalyst of Example 3 under different test conditions.

a) In a modification of the standard test conditions, testing was carried out at a temperature of 180° C., under a pressure of 5 bar and with a catalyst load of 14 l/(g(cat)*h).

b) In a modification of the standard test conditions, testing was carried out at a temperature of 180° C. and under a pressure of 5 bar.

### Example 7

Example 7 describes the preparation and use of a catalyst in which a molybdenum compound has been applied to a previously prepared silicon oxide carrier by impregnation from aqueous solution, with the subsequent application of gold by a deposition-precipitation method.

For the preparation of the carrier, 7120 mg (46.8 mmol.) of tetramethoxysilane and 708 mg of methyltrimethoxysilane (5.2 mmol.) were placed in a PE beaker together with 7176 mg (156 mmol.) of ethanol. 1638 mg (26 mmol.) of HNO₃ in a total of 468 mg (106 mmol.) of H₂O were then added, and the batch was stirred until gelling occurred. After an ageing time of 12 hours, the gel was dried at 60° C./200 mbar, comminuted in a mortar and then calcined for 4 hours at 120° C.

For the application of the molybdenum by impregnation (target content 5% Mo), 1.5 g of carrier material were added to 1.16 ml of an aqueous solution of MoO₃ (9 wt. % MoO₃), which had been prepared beforehand by dissolution with NH₄OH at pH 8, at pH 6.3 (adjustment by addition of HNO₃) and stirring was carried out. After being allowed to react for 30 minutes, the batch was dried at 100° C./200 mbar and calcined for 15 hours at 300° C.

The molybdenum-containing carrier was introduced into a centrifuge vessel, and 7.5 ml of an aqueous solution of HAuCl₄ (concentration 1 g Au/l) were added. Sodium hydroxide solution (conc.=0.1 mol./l) was added, with stirring, until a constant pH value of 8.0±0.1 was maintained over a period of several minutes. 7.6 ml of an aqueous sodium citrate solution (concentration=0.015 mol./l, adjusted to pH 8) were then added, and stirring was carried out for 10 minutes. After centrifugation, washing was carried out 3 times using 20 ml of deionized water each time, followed by centrifugation. The powder loaded with gold was dried at 60° C./200 mbar and then tempered for 4 hours at 300° C. under an N₂ atmosphere. The catalyst was tested under standard conditions.

### Example 8

Example 8 describes the preparation and use of a catalyst in which gold has been applied to a commercially available Nb₂O₅ carrier by a deposition-precipitation method, with the subsequent application of a molybdenum compound by means of impregnation from aqueous solution. The application of molybdenum and gold to the commercially available carrier (1.5 g) was carried out analogously to Example 7. The catalyst was tested under standard conditions.

### Comparison Example 1 (in accordance with Example 7 of EP-A 1 125 632)

For the preparation of an Au/Mo catalyst, 2.92 ml of ethanol were mixed with 3298 mg of tetraethyl orthosilicate, and 227.5 mg of MoCl₅ were added. 1.67 g of HNO₃ dissolved in 0.600 ml of H₂O were added to that mixture, and the sample was mixed until gelling occurred. The sample was then dried, ground and heated for 24 hours at 350° C. For the purposes of loading with gold, 1 g of the Mo-containing carrier was suspended in 20 ml of water, 0.2 g of HAuCl₄ was added to the mixture, and mixing was carried out for one hour. 10 ml of 0.015 molar sodium citrate solution were then added, and the system was mixed for a further one hour. The moist powder was separated off, washed several times with distilled water in order to remove chlorine, dried overnight at 100° C. and 200 mbar and finally calcined at 350° C.

500 mg of the catalyst were tested in a gas reaction cell at 100° C., with a gas composition of 5.78% propene, 75.65% hydrogen, 4.81% oxygen and 13.76% nitrogen and a flow rate of 3500 ml/(g(cat)*h).

### Comparison Example 2 (in accordance with Example 1 of DE-A 199 59 525)

For the preparation of an Au catalyst containing Ti, 1.9 g of a 0.1 n p-toluenesulfonic acid solution in water were added to 10.1 g of methyltrimethoxysilane and 15 g of ethanol, and the mixture was stirred for 2 hours. 1.46 g of tetrabutoxytitanium were then added slowly, stirring was carried out for 30 minutes, a solution of 5.6 g of triethoxysilane was added, followed by stirring for a further 30 minutes, a mixture of 1.23 g of a 0.1 n solution of p-toluenesulfonic acid in water was added, with stirring, and the whole was finally allowed to stand. The batch reached the gel point after about 7 minutes. After an ageing time of 24 hours, the gel was comminuted in a mortar and dried for 8 hours at 120° C. in the air. 5.4 g of sol-gel material were impregnated with a solution consisting of 540 mg of a 1% methanolic Au solution, which had been made up to 2.8 g with methanol, and the macroscopically dry material was dried for 4 hours at room temperature and then tempered for 2 hours at 400° C. under a nitrogen atmosphere. 500 mg of the catalyst were tested in a tubular reactor at 140° C. with a molar gas composition of N₂/H₂/O₂/C₃H₆:14/75/5/6 and with a load of 3.0 l/(g(cat)*h).

The test results for the catalysts of the Examples are summarized in Table 1.

TABLE 1
Test results for the Examples

| | | | | | Deactivation |
|---|---|---|---|---|---|
| | Maximum PO | | | Maximum PO | [% loss in |
| | yield [%] | | | productivity | yield per |
| | (% propene | | Selectivity | [mg PO/ | 10 hours] |
| Example | in the feed) | | [%] | (gcat*h)] | (running time) |
| 1 | 0.75 | (20) | 94 | 10.4 | 0.66 (500 h) |
| 2 | 0.84 | (20) | 93 | 11.3 | 1.10 (430 h) |
| 3 | 0.98 | (20) | 92 | 13.3 | 0.46 (825 h) |
| 4 | 0.86 | (20) | 92 | 12.1 | 0.04 (3300 h) |
| 5 | 0.64 | (20) | 86 | 9.0 | 0.27 (600 h) |
| 6a | 0.91 | (20) | 93 | 68.2 | 0.43 (330 h) |
| 6b | 1.64 | (20) | 94 | 18.1 | 0.23 (550 h) |
| 7 | 1.1 | (20) | 95 | 15 | 0.27 (1100 h) |
| 8 | 0.78 | (20) | 95 | 10.6 | n.d. |
| Comp. 1 | 0.01 | (5.8) | 72.15 | 0.05 | n.i. |
| Comp. 2 | 8 | (6) | 95 | 37.2 | 0.30 (240 h) |

(PO = propene oxide,
feed = gas stream to which the bulk catalyst is subjected,
cat = catalyst,
n.d. = not determined,
n.i. = no information,
Comp. = comparison example)

In the last column of Table 1 (heading: Deactivation), the percentage loss in yield per 10 hours' operating time of the catalyst is given. The figure indicated in brackets is the operating time, in hours, after which the loss in yield in % per 10 hours operating time was measured. The longer that operating time, the more reliable and more meaningful the value of the loss in yield per 10 hours for an assessment of performance.

Selectivity is defined as the proportion of the sum of the carbon atoms of the converted propene that is contained in the resulting propene oxide.

The Examples can be evaluated as follows. The listed data are all parameters for assessing the performance of the tested catalysts. In order to evaluate the overall performance, all the values must be considered together, but it may also be useful to pick out individual aspects in order to indicate a positive trend. That is clearly possible in that all the catalysts according to the invention perform considerably better in all available parameters. In comparison with the titanium-containing catalysts (Comparison Example 2), attention should be drawn substantially to an improvement in the lifetime behavior (under normal pressure and elevated pressure). This should demonstrate that the principal disadvantage of titanium-containing catalysts, namely their rapid deactivation, is less strongly pronounced in the case of the molybdenum-containing catalysts. In respect of individual parameters, the titanium-containing catalyst, in particular in respect of productivity at normal pressure, is still superior.

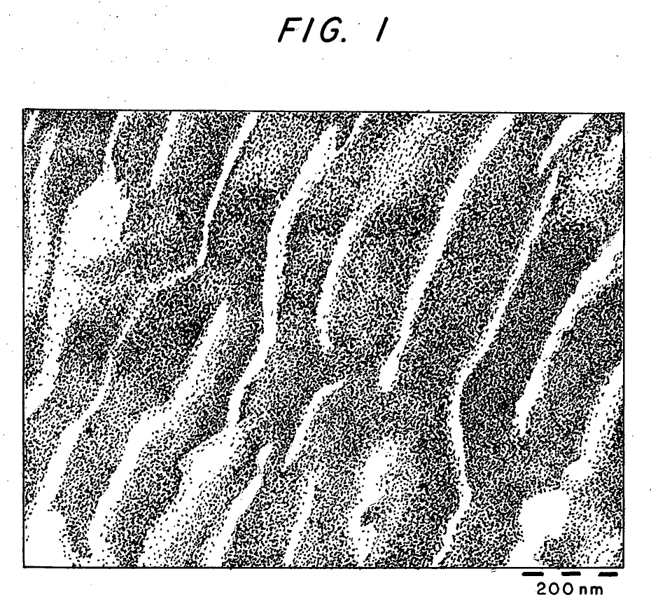

Examination of the catalysts by transmission electron microscopy (TEM)

Where a typical Au/Mo catalyst according to the invention was examined by transmission electron microscopy (see FIG. 1), the presence of nanostructured Au particles of the order of magnitude of <20 nm could clearly be demonstrated.

FIG. 1 shows the TEM image of a catalyst obtained according to Example 7, on a scale of 100,000:1. The gold particles are visible as dark spots.

Examination of the catalysts by X-ray absorption spectrometry (XAS)

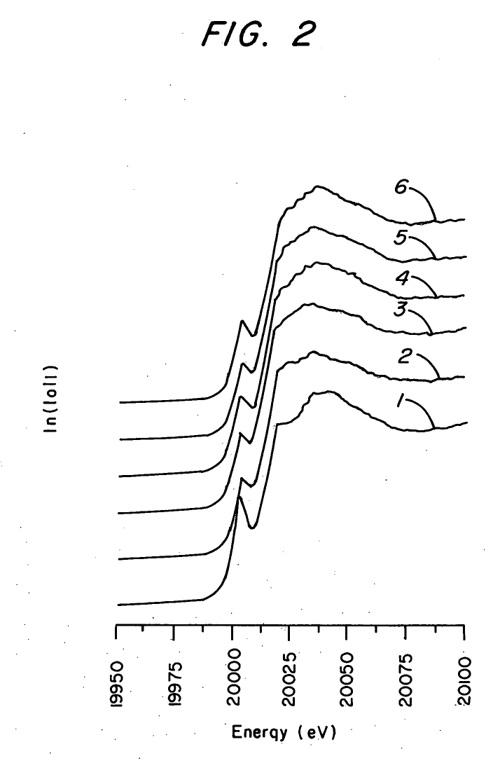

FIG. 2 shows the XAS spectra of four catalysts obtained according to the Examples (1=Na₂MoO₄, comparison substance tetrahedral molybdenum, 2=ammonium heptamolybdate, comparison substance octahedral molybdenum, 3=catalyst according to Example 5, 4=catalyst according to Example 2, 5=catalyst according to Example 1 before the reaction, 6=catalyst according to Example 1 after the reaction). The energy of the X-radiation in electron volts (eV) is plotted on the horizontal axis. The absorption of the X-radiation (natural logarithm (irradiated intensity Iₒ/intensity I detected behind the sample)) is plotted on the vertical axis. Because several spectra have been superposed, scaling of the y-axis is not possible. However, the spectra have been standardized so that they are directly comparable.

In order to examine the Mo coordination and the oxidation number, X-ray absorption spectra (XAS, see FIG. 2) of typical Au- and Mo-containing catalysts according to the invention were recorded at the Mo-K edge. The desired information could be derived from the XANES (X-ray absorption near edge structure) and EXAFS (extended X-ray absorption fine structure) regions of the spectrum.

In the XANES region of the spectrum, the coordination number of the molybdenum in the catalysts according to the invention could be derived by comparison with tetrahedral (Na₂MoO₄) and octahedral (ammonium heptamolybdate) model substances. The pre-edge absorption (see FIG. 2) is a marked indication of octahedral coordination (strong similarity with ammonium heptamolybdate).

The EXAFS analysis of the X-ray absorption spectrum of typical Au/Mo catalysts according to the invention (catalysts obtained according to the above-mentioned Examples 1, 2 and 5 were studied) confirms the presence of predominantly octahedrally coordinated Mo centers in oxidation state +VI, the experimental data indicating two short Mo(IV)—O bond distances of 1.7 angstrom and four long Mo(VI)—O distances of from 1.9 to 2.3 angstrom (distorted octahedron).

Although the invention has been described in detail in the foregoing for the purpose of illustration, it is to be understood that such detail is solely for that purpose and that variations can be made therein by those skilled in the art without departing from the spirit and scope of the invention except as it may be limited by the claims.

What is claimed is:

1. A catalyst containing gold in elemental or bound form and molybdenum in oxidation state +VI.

2. The catalyst according to claim 1, wherein the molybdenum is present in octahedral coordination.

3. A process for the preparation of the catalyst according to claim 1, comprising the steps of:
applying gold in elemental or bound form to a carrier matrix,
applying molybdenum to the carrier matrix, and
tempering the carrier matrix loaded with gold and molybdenum at a temperature of from 200 to 500° C., with the substantial exclusion of oxygen.

4. The catalyst obtained by the process according to claim 3.

5. In a process for the oxidation of a hydrocarbon containing at least one double bond to an epoxide, the improvement comprising reacting the hydrocarbon with an oxygen source in the presence of a hydrogen source and in the presence of the catalyst according to claim 1.
